(19) United States
(12) Patent Application Publication
(10) Pub. No.: US 2009/0194123 A1
(43) Pub. Date: Aug. 6, 2009

(54) HAIR RELAXING KIT AND PROCESS COMPRISING AT LEAST ONE TERTIARY AMINE

(75) Inventors: Gerard Malle, Villiers S/Morin (FR); Damien Lavergne, Paris (FR); Xavier Radisson, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'Oreal S.A.

(21) Appl. No.: 12/365,651

(22) Filed: Feb. 4, 2009

Related U.S. Application Data
(62) Division of application No. 10/989,571, filed on Nov. 17, 2004, now abandoned.
(60) Provisional application No. 60/562,219, filed on Apr. 15, 2004.

(30) Foreign Application Priority Data
Nov. 18, 2003 (FR) 0350850

(51) Int. Cl. A45D 7/06; A61K 8/41; A61Q 5/04; C07C 211/01

(52) U.S. Cl. 132/205; 424/70.2; 564/463

(57) ABSTRACT

Disclosed herein is a ready-to-use cosmetic composition for relaxing keratin fibers comprising, as relaxing agent, at least one secondary or tertiary amine. Further disclosed herein are a kit comprising at least two compartments to be placed in contact to form the ready-to-use composition, and a process using this composition.

This application claims benefit of U.S. Provisional Application No. 562,219, filed Apr. 15, 2004.

The present disclosure relates to a ready-to-use cosmetic composition for relaxing keratin fibers, comprising at least one relaxing agent chosen from secondary and tertiary amines. The disclosure is also directed to a kit comprising at least two compartments to be placed in contact to form the ready-to-use composition, and also to a process using the same.

As used herein, the term “keratin fibers” means fibers of human or animal origin such as head hair, other body hairs, the eyelashes, wool, angora, cashmere or fur. Although the present disclosure is not limited to particular keratin fibers, in one embodiment, the keratin fibers are head hair.

As used herein, the terms “relaxing” and “straightening” include the relaxing, smoothing-out or decurling of Caucasian, Asiatic, North African or African hair.

Further, as used herein, the term “at least one amine chosen from secondary and tertiary amines” comprises amines and their organic or minerals salts which do not contain hydroxide ions in their chemical formula. The term can also mean a compound capable of accepting a proton, and deriving from the substitution of two or three hydrogen atoms of ammonia with carbon-containing radicals singly bonded to the nitrogen atom.

In the present disclosure, the term “between x % and y %” means ranging from x % to y %, wherein the limits x and y are included.

Two techniques may be used to permanently reshape the hair. They are based on breaking the disulfide bonds present in keratin (cystine):
The first technique comprises, in a first step, opening the disulfide bonds with a composition comprising at least one reducing agent, and then, after having, in some instances, rinsed the hair, a second step of reconstituting the disulfide bonds by applying to the hair, which has been placed under tension beforehand with curlers or the like or shaped or smoothed out by other means, an oxidizing composition also known as a fixing composition, giving the head of hair the desired shape. This first technique may make it possible either to make the hair wavy or relaxed, i.e., to decurl or to straighten.
In a second technique, the first step comprises performing a lanthionization operation, using a composition comprising a base belonging to the hydroxide family, i.e., at least one hydroxide base. This second technique can lead to replacement of the disulfide bonds (—CH₂—S—S—CH₂—) with lanthionine bonds (—CH₂—S—CH₂—). The lanthionization operation may involve two consecutive chemical reactions:
The first reaction comprises a beta-elimination on the cystine caused by a hydroxide ion, leading to the breaking of this bond and the formation of dehydroalanine.

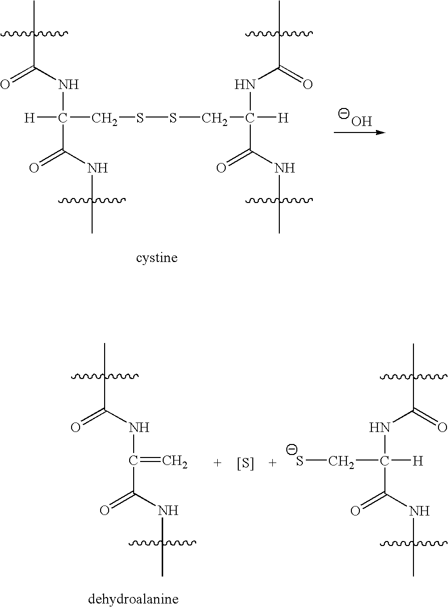

The second reaction is a reaction of the dehydroalanine with a thiol group. Specifically, the double bond of the dehydroalanine formed is a reactive double bond. It can react with the thiol group of the cysteine residue that has been released to form a new bond, referred to as a lanthionine bridge or bond or residue.

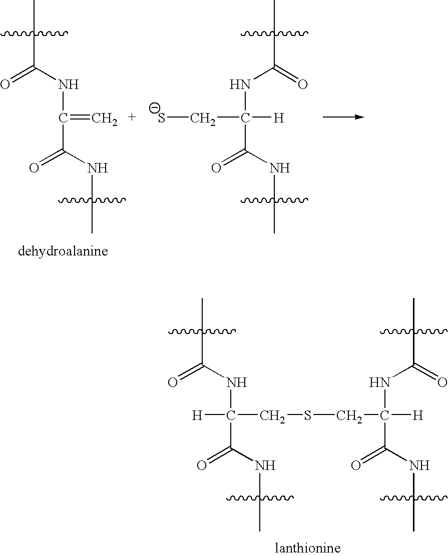

In comparison with the first technique, the second technique, i.e., the lanthionization operation, does not require a fixing step, since the formation of the lanthionine bridges is irreversible. As such, the reaction may take place in a single step and may make it possible either to make the hair wavy or relaxed, i.e., decurl or straighten the hair. The lanthionization operation, however, is mainly used to relax naturally frizzy or curly hair.

With regard to the first technique, the reducing compositions typically used for the first step of a permanent-waving or relaxing operation comprise at least one reducing agent chosen from thiols, sulfites, and bisulfites. The at least one reducing agent is generally used in essentially aqueous medium at concentrations ranging from 0.5 to 1M to obtain good opening of the disulfide bonds. Among the thiols, those commonly used are thioglycolic acid, cysteamine, glyceryl monothioglycolate, thiolactic acid and cysteine. Thioglycolic acid is known to be efficient at reducing the disulfide bonds of keratin at alkaline pH, such as in the form of ammonium thioglycolate, and is the product most commonly used in permanent-waving (hair waving). However, it has been found that thioglycolic acid should be used in a sufficiently basic medium (in practice at a pH of between 8.5 and 9.5), if it is desired to obtain curling of sufficient intensity. Besides the drawback of releasing an unpleasant odor requiring the use of more or less efficient fragrances to mask the odors, the use of a thiol at an alkaline pH may also lead to degradation of the fiber such as, impairment of the artificial colorations.

Sulfites or bisulfites may be used for relaxing. They have drawbacks similar to thiols, with lower efficacy.

Thiols and sulfites (or bisulfites) also have the drawback of having poor stability in aqueous solution.

In general, the durability of the reshaping effects obtained with thiols and/or sulfites by reduction of disulfides followed by fixing may be considered to be very much inferior to that which may be obtained via the lanthionization technique.

For the second technique, the compositions generally used to perform the lanthionization comprise at least one hydroxide base, such as sodium hydroxide, guanidinium hydroxide and lithium hydroxide. These lanthionization active agents, which make it possible to open the disulfide bonds via a beta-elimination mechanism, are generally used in a water-in-oil emulsion at concentrations ranging from 0.4M to 0.6M, by leaving them to act generally for 10 to 15 minutes at room temperature. Sodium hydroxide is the agent most commonly used, although guanidinium hydroxide is now the preferred compound for many compositions. These two hydroxides, i.e., sodium hydroxide and guanidinium hydroxide, are the two main agents used for relaxing or decurling naturally frizzy/curly hair. They have several advantages over ammonium thioglycolate and sulfites, such as an absence of unpleasant odor, the fact that only one implementation step is required (shorter treatment time), and much greater durability and efficacy of the reshaping of the hair.

However, these hydroxide bases have the major drawback of being caustic. This causticity may affect the scalp by causing irritation that on occasion may be severe. This may be partially overcome by the prior application to the scalp of a greasy protective cream often referred to as “base” or “base cream”, wherein the word “base” as used here does not have the meaning of a basic agent in the chemical sense. When the protective cream is combined with the hydroxide component in a single composition, this is generally referred to as a “no-base” composition, as opposed to the above name. This “no-base” technology, may, for example, be used, in part because it is more convenient.

The causticity of the hydroxides may also affect the state of the hair by, on the one hand, giving it a coarse feel and on the other hand, making it much more fragile, this fragility may possibly go as far as fraying, breaking or even dissolution of the hair if the treatment is prolonged. In certain cases, hydroxides may also cause decoloration of the natural color of the hair.

Formulations comprising sodium hydroxide are generally referred to as “lye relaxers” and those not containing it are known as “no-lye relaxers.”

The main “no-lye” relaxing formulations use guanidinium hydroxide. Since guanidinium hydroxide may be unstable, it is generated extemporaneously by mixing guanidine carbonate and a source of very sparingly soluble hydroxide such as, calcium hydroxide. The reaction between these two compounds leads to the formation of guanidinium hydroxide and calcium carbonate, which precipitates in the composition. The presence of this precipitate may make the final rinsing of the hair much more difficult and may leave on the hair and scalp mineral particles that can give it a coarse feel and an unaesthetic appearance resembling dandruff. The recent success of guanidinium hydroxide (“no-lye”) over sodium hydroxide (“lye”) appears to arise from better relaxing efficacy and better skin tolerance. However, these technologies using the hydroxide bases remain very aggressive for the hair and the scalp and require very strict control of the application time to avoid excessive irritation and impairment of the hair that may go as far as breaking. This aggressiveness arising from the causticity of hydroxides is just the reason for these compositions for the lanthionization of the hair not to be used for permanent-waving (hair waving), but instead to be generally reserved for relaxing (hair straightening or hair relaxing).

Furthermore, hydroxides are known to be good agents for hydrolysing amide functions (see for example, March's Advanced Organic Chemistry, 5th Edition, Wiley Interscience, New York, “Hydrolysis of Amides” pages 474 et seq), which may thus lead to breaking of the peptide bonds by direct nucleophilic attack. It is thus possible that the impairments observed in the case of the hair and keratin materials in the broad sense are largely due to partial hydrolysis of the amide bonds of keratin.

There is thus a real need, in relaxing, for compositions that are less aggressive to the hair and the skin.

Various studies have been performed with a view to simultaneously overcoming at least one of the drawbacks of reducing agents (the first technique) and/or of hydroxides (the second technique).

Thus, to replace thioglycolic acid, many reducing agents have been proposed, but thioglycolic acid in its ammonium thioglycolate form remains both the reference compound and the compound most widely used in cosmetic formulations, both for shaping and for relaxing and straightening the hair.

To replace sodium hydroxide or guanidinium hydroxide and to improve the skin tolerance, it has been proposed, in U.S. Pat. No. 4,530,830, to use a composition based on quaternary ammonium hydroxides. However, these compositions are not entirely satisfactory, either in terms of relaxing or in cosmetic terms.

More generally, many publications describe the combined use of hydroxides, acting as lanthionization active agents, with certain additives generally acting to protect the hair.

Specifically, without using novel lanthionization active agents, the improvements proposed relate mainly to the use of additives to reduce the damage caused on the hair by hydroxides. Reference may be made, for example, to:
International Patent Application No. WO 2002/003937, which describes a composition comprising C3-C5 monosaccharides,
International Patent Application No. WO 2001/064171, which describes a composition comprising complexing agents,
U.S. Pat. No. 5,641,477, which describes a composition comprising a hydrogenated starch hydrolysate,
International Patent Application No. WO 02/085317, which describes a composition comprising organic nucleophiles, which react during the second step with the dehydroalanine formed with hydroxides, to give new bridges,
U.S. Pat. No. 5,679,327, which describes a relaxing composition comprising three active ingredients for relaxing, namely, an alkaline hydroxide, an alkaline-earth metal hydroxide and a nitrogenous organic base, wherein each of the ingredients is present in the composition in a proportion that would be insufficient to effect relaxing if it were used at this same concentration without the other two active ingredients. In other words, there may be a synergism between the three ingredients, these ingredients together resulting in relaxing the keratin fibers.

Although all these proposals can lead to more or less marked improvements, they do not make it possible to sufficiently reduce the damage caused by the actual causticity of the hydroxides.

As indicated previously, the use of reducing agents can lead to mediocre durability of the relaxing or decurling and the use of hydroxides, on account of their causticity, may limit their use in the relaxing field.

After extensive studies, it has now been discovered, entirely surprisingly and unexpectedly, that the first step of the lanthionization process can be performed with at least one amine chosen from secondary and tertiary amines. Excellent results in terms of relaxing and cosmetic and mechanical properties of the hair can thus be obtained.

Thus, disclosed herein is a cosmetic composition comprising, in a cosmetically acceptable medium, at least one amine chosen from secondary and tertiary amines, wherein the cosmetically acceptable medium and the at least one amine are chosen such that the at least one amine is reactive with the cystines of the keratin fibers, via a beta-elimination reaction, to produce dehydroalanine and lead to the formation of lanthionine, and thus, to relax the keratin fibers in less than 60 minutes.

In one embodiment, the relaxing time is less than 50 minutes, for example, less than 40 minutes.

The expression “secondary or tertiary amines,” which may be used as beta-elimination active agents resulting in lanthionization, means any derivative resulting from the substitution of two or three hydrogen atoms of ammonia with carbon-containing radicals singly bonded to the nitrogen atom, not containing in its formula hydroxide ions, and being capable of accepting a proton. These secondary or tertiary amines are, for example, chosen from cyclic secondary or tertiary amines such as cyclic tertiary amines.

In one embodiment of the disclosure, a tertiary amine which may be used as a beta-elimination active agent resulting in lanthionization, is 1,4,7-trimethyl-1,4,7-triazacyclononane (Registry Number [96556-05-7]) and a secondary amine which may be used as a beta-elimination active agent resulting in lanthionization is 1,4,7-triazacyclononane (Registry Number [4730-54-5]).

In the compositions disclosed herein intended for a hair relaxing, decurling or straightening process, the at least one secondary or tertiary amine is, for example, present in a molar concentration ranging from 0.1M to 4M, which corresponds to concentrations ranging from 1% to 80% by weight, relative to the total weight of the composition, and further for example, in a concentration ranging from 0.2M to 4M, which corresponds to concentrations ranging from 2% to 80% by weight, relative to the total weight of the composition.

The pH of the compositions as disclosed herein ranges, for example, from 9.6 to 14, such as from 11 to 13.

In one embodiment, in the compositions of the present disclosure, the at least one secondary or tertiary amine is the only relaxing active agent.

The compositions according to the present disclosure may also comprise known reducing agents chosen from, for example, thioglycolic acid and thiolactic acid and ester and amide derivatives thereof, such as glyceryl monothioglycolate, cysteamine and its C1-C4 acyl derivatives, such as N-acetylcysteamine and N-propionylcysteamine, cysteine, N-acetylcysteine, thiomalic acid, pantethine, 2,3-dimercaptosuccinic acid, sulfites and bisulfites of an alkali metal or an alkaline-earth metal, the N-(mercaptoalkyl)-ω-hydroxyalkylamides described in European patent application EP-A-354 835, the N-mono- or N,N-dialkylmercapto-4-butyramides described in European patent application EP-A-368 763, the aminomercaptoalkylamides described in European patent application EP-A-432 000, the N-(mercaptoalkyl)succinamic acid and N-(mercaptoalkyl)succinimide derivatives described in European Patent Application No. EP-A-465 342, the alkylamino mercaptoalkylamides described in European Patent Application No. EP-A-514 282, the azeotropic mixture of 2-hydroxypropyl thioglycolate and of (2-hydroxy-1-methyl)ethyl thioglycolate described in French Patent Application No. FR-A-2 679 448, the mercaptoalkylaminoamides described in French Patent Application No. FR-A-2 692 481, the N-mercaptoalkylalkanediamides described in European Patent Application No. EP-A-653 202 and the formamidinesulfinic acid derivatives described in International Patent Application No. PCT/US 01/43124.

When the compositions disclosed herein comprise at least one reducing agent, the at least one reducing agent is, for example, present in a maximum concentration of 20% by weight, such as ranging from 0.1% to 10% by weight, relative to the total weight of the composition.

The compositions as disclosed herein may also comprise known hydroxides chosen from, for example, alkali metal hydroxides and alkaline-earth metal hydroxides and transition metal hydroxides and organic hydroxides, such as sodium hydroxide, potassium hydroxide, lithium hydroxide, rubidium hydroxide, caesium hydroxide, francium hydroxide, beryllium hydroxide, magnesium hydroxide, calcium hydroxide, strontium hydroxide, barium hydroxide, molybdenum hydroxide, manganese hydroxide, zinc hydroxide, cobalt hydroxide, cadmium hydroxide, cerium hydroxide, lanthanum hydroxide, actinium hydroxide, thorium hydroxide, aluminium hydroxide, guanidinium hydroxide, and quaternary ammonium hydroxides.

When the compositions of the disclosure comprise at least one hydroxide, this at least one hydroxide is, for example, present in a concentration ranging from 0.01% to 3.5% by weight, such as ranging from 0.05% to 1.5% by weight, relative to the total weight of the composition.

In one further embodiment of the compositions according to the disclosure, these compositions comprise 0% of a hydroxide base, chosen from, for example, alkali metal hydroxides and alkaline-earth metal hydroxides and transition metal hydroxides, and organic hydroxides.

In one embodiment, the compositions as disclosed herein comprise from 0% to 100%, such as from 0% to 80% and, further for example, from 0% to 50% of water by weight, relative to the total weight of the composition.

According to another embodiment, the basic compositions also comprises at least one surfactant chosen from nonionic, anionic, cationic and amphoteric surfactants, and mention may be made, for example, of alkyl sulfates, alkylbenzene sulfates, alkyl ether sulfates, alkyl sulfonates, quaternary ammonium salts, alkylbetaines, oxyethylenated alkylphenols, fatty acid alkanolamides, oxyethylenated fatty acid esters, and other nonionic hydroxypropyl ether surfactants.

When the composition as disclosed herein comprises at least one surfactant, this at least one surfactant is generally present in a maximum concentration of 30% by weight, such as from 0.5% to 10% by weight, relative to the total weight of the composition.

In order to improve the cosmetic properties of the hair or to attenuate or avoid the hair's degradation, the presently disclosed composition may also comprise at least one treating agent chosen from treating agents of cationic, anionic, non-ionic, and amphoteric nature.

Among the treating agents mention may be made, for example, to those described in French Patent Nos. 2 598 613 and 2 470 596. It may also be possible to use, as treating agents, volatile or non-volatile, linear or cyclic silicones and mixtures thereof, polydimethylsiloxanes, quaternized polyorganosiloxanes, such as those described in French Patent Application No. 2 535 730, polyorganosiloxanes comprising aminoalkyl groups modified with alkoxycarbonylalkyl groups, such as those described in U.S. Pat. No. 4,749,732, polyorganosiloxanes, such as the polyoxyalkyl polydimethylsiloxane copolymer of the Dimethicone Copolyol type, a polydimethylsiloxane comprising stearoxy end groups (stearoxy dimethicone), a dialkylammonium acetate polydimethylsiloxane or a polydimethylsiloxane polyalkylbetaine copolymer described in British Patent No. 2 197 352, polysiloxanes organomodified with mercapto or mercaptoalkyl groups, such as those described in French Patent No. 1 530 369 and in European Patent Application No. 295 780, and also silanes, such as stearoxytrimethylsilane.

The compositions according to the disclosure herein may also comprise at least one other treating ingredient, such as cationic polymers, for instance those used in the compositions of French Patent Nos. 79/32078 (2 472 382) and 80/26421 (2 495 931) or cationic polymers of the ionene type, such as those used in the compositions of Luxembourg Patent No. 83703, basic amino acids (such as lysine and arginine) and acidic amino acids (such as glutamic acid and aspartic acid), peptides and derivatives thereof, protein hydrolysates, waxes, swelling agents and penetrating agents and agents for reinforcing the efficacy of the reducing agent, such as the SiO₂/PDMS (polydimethylsiloxane) mixture, dimethylisosorbitol, urea and its derivatives, pyrrolidone, N-alkylpyrrolidones, thiamorpholinone, alkylene glycol and dialkylene glycol alkyl ethers, for instance, propylene glycol monomethyl ether, dipropylene glycol monomethyl ether, ethylene glycol monoethyl ether and diethylene glycol monoethyl ether, C3-C6 alkanediols, for instance, propane-1,2-diol, propane-1,3-diol and butane-1,2-diol, glycerol, 2-imidazolidinone, and also other compounds such as fatty alcohols, lanolin derivatives, active ingredients, such as pantothenic acid, agents for preventing hair loss, antidandruff agents, thickeners, suspending agents, sequestering and complexing agents, opacifiers, sunscreens, fragrances, and preserving agents.

As used herein, the term “cosmetically acceptable medium” is known to one of ordinary skill in the art, and may comprise, for example, water and/or at least one organic solvent.

The compositions according to disclosure are, for example, in the form of a thickened cream so as to hold the hair as stiff as possible. These creams can be made in the form of “heavy” emulsions, for example, based on glyceryl stearate, glycol stearate, self-emulsifying waxes, or fatty alcohols.

Liquids or gels comprising thickeners, such as carboxyvinyl polymers or copolymers that “stick” the hairs together and hold them in a smooth position during the leave-in time, may also be used.

The compositions according to disclosure herein may also comprise at least one adjuvant chosen from, for example, silicones in soluble, dispersed and microdispersed form, nonionic, anionic, cationic and amphoteric surfactants, ceramides, glycoceramides and pseudoceramides, vitamins and provitamins including panthenol, plant, animal, mineral and synthetic oils, waxes other than ceramides, glycoceramides and pseudoceramides, water-soluble and liposoluble, silicone-based or non-silicone-based sunscreens, nacreous agents and opacifiers, sequestering agents, plasticizers, solubilizers, acidifying agents, mineral and organic thickeners, antioxidants, hydroxy acids, penetrating agents, fragrances and preserving agents. Among the solubilizers that may be mentioned, for example, are lower alcohols, for instance ethanol, propanol or isopropanol.

The present disclosure also relates to a kit comprising at least two compartments, wherein one of the compartments comprises a composition (i) comprising, in a cosmetically acceptable medium, at least one amine chosen from secondary and tertiary amines, wherein said at least one amine is reactive with the cystines of keratin fibers via a beta-elimination reaction to produce dehydroalanine and lead to the formation of lanthionine, to relax keratin fibers in less than 60 minutes.

In one embodiment, the kit also comprises an additional composition (ii) for caring for, conditioning, making up, removing makeup from, protecting, cleansing and/or washing keratin fibers.

The compositions of the kits as disclosed herein are packaged in separate compartments, containers or devices, optionally accompanied by suitable, identical or different application tools, such as fine brushes, coarse brushes and sponges.

This disclosure also relates to a process for relaxing keratin fibers using a cosmetic composition comprising, in a cosmetically acceptable medium, at least one amine chosen from secondary and tertiary amines, wherein the cosmetically acceptable medium and the at least one amine are chosen such that the at least one amine reacts with the cystines of the keratin material, via a beta-elimination reaction to produce dehydroalanine and lead to the formation of lanthionine, to relax the keratin material in less than 60 minutes.

In one further embodiment, in the process as disclosed herein, the relaxing time is less than 50 minutes, such as less than 40 minutes.

In the hair relaxing, decurling, or straightening process as disclosed herein, the composition according to the present disclosure is applied, for example, to keratin fibers, such as hair, and the keratin fibers are then subjected to mechanical reshaping allowing them to be given a new shape, via an operation of smoothing-out via a large-toothed comb, with the back of a comb or by hand. After a leave-in time ranging from 5 to 60 minutes such as, from 5 to 40 minutes, the keratin fibers are smoothed out again and then rinsed thoroughly.

After applying the composition as disclosed herein, the keratin fibers, e.g., a head of hair, may, for example, be subjected to a heat treatment by heating to a temperature ranging from 30° to 60° C. In practice, this operation may be performed using a hairstyling hood, a hair drier, an infrared ray dispenser and/or other standard heating devices.

It is possible to use, as a method of both heating and smoothing out the keratin fibers, such as hair, a hot iron at a temperature ranging from 60° to 220° C. such as, from 120° and 200° C.

Further disclosed herein is the use of at least one secondary or tertiary amine as an active agent for relaxing keratin fibers.

The present disclosure also relates to an active agent for relaxing keratin fibers, by a beta-elimination reaction producing dehydroalanine and leading to the formation of lanthionine, comprising at least one secondary or tertiary amine.

Other than in the operation examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that numerical ranges and parameters setting forth the broad scope of the disclosure are approximations, the numerical values set forth in the specific examples are reported to as precisely as possible. Any numerical value, however, inherently contains certain errors necessarily resulting from the standard deviation found in their respective testing measurements.

The invention may be understood more clearly with the aid of the non-limiting examples that follow.

### EXAMPLE 1

A simplified relaxing composition was prepared, comprising 1,4,7-trimethyl-1,4,7-triazacyclononane at a concentration of 1M in water, as relaxing active agent. The pH of the composition was 13.1. This composition was applied to naturally frizzy/curly North African hair for 30 minutes at a temperature of 50° C. The hair was efficiently relaxed, easy to comb, and felt soft.

### EXAMPLE 2

A simplified relaxing composition was prepared, comprising 1,4,7-trimethyl-1,4,7-triazacyclononane at a concentration of 2M in water, as relaxing active agent. The pH of the composition was 13.3. This composition was applied to naturally frizzy/curly African hair for 40 minutes at a temperature of 50° C. The hair was relaxed, easy to comb and style, and felt soft.

### EXAMPLE 3

A simplified relaxing composition was prepared, comprising 1,4,7-triazacyclononane at a concentration of 1.5M in water, as relaxing active agent. The pH of the composition was 12.7. This composition was applied to naturally frizzy/curly North African hair for 35 minutes at a temperature of 50° C. The hair was relaxed, easy to comb and style, and felt soft.

1-13. (canceled)

14. A kit comprising at least two compartments, wherein one of the compartments comprises a composition (i) comprising, in a cosmetically acceptable medium, at least one amine chosen from tertiary amines, wherein said at least one amine is reactive with the cystines of keratin fibers via a beta-elimination reaction to produce dehydroalanine and lead to the formation of lanthionine, to relax keratin fibers in less than 60 minutes.

15. The kit according to claim 14, further comprising an additional composition (ii) for caring for, conditioning, making up, removing makeup from, protecting, cleansing and/or washing keratin fibers.

16. A process for relaxing keratin fibers comprising
applying to the keratin fibers a cosmetic composition comprising, in a cosmetically acceptable medium, at least one amine chosen from tertiary amines, wherein the cosmetically acceptable medium and the at least one amine are chosen such that the at least one amine reacts with the cystines of the keratin fibers, via a beta-elimination reaction, producing dehydroalanine and leading to the formation of lanthionine, to relax the keratin fibers in less than 60 minutes.

17. The process according to claim 16, wherein the keratin fibers are head hair.

18. The process according to claim 16, further comprising, after applying the composition, heating the keratin fibers to a temperature ranging from 30° to 60° C.

19. The process according claim 16, further comprising heating and smoothing out the keratin fibers with a hot iron at a temperature ranging from 60° and 220° C.

20. The process according to claim 19, wherein the hot iron is at a temperature ranging from 120° and 200° C.

21. The process according to claim 16, wherein the relaxing time is less than 50 minutes.

22. The process according to claim 21, wherein the relaxing time is less than 40 minutes.

23. An active agent for relaxing keratin fibers comprising at least one amine chosen from tertiary amines, wherein the at least one amine is reactive with the cystines of keratin fibers via a beta-elimination reaction to produce dehydroalanine and lead to the formation of lanthionine, to relax the keratin fibers.

24. The process according to claim 16, wherein the at least one amine is present in a molar concentration ranging from 0.1 M to 4 M.

25. The process according to claim 24, wherein the at least one amine is present in a molar concentration ranging from 0.2 M to 4 M.

26. The process according to claim 16, wherein the pH of the composition ranges from 9.6 to 14.

27. The process according to claim 26, wherein the pH of the composition ranges from 11 to 13.

28. The process according to claim 16, wherein the composition comprises 0% of a hydroxide base.

29. The process according to claim 16, wherein the composition comprises from 0% to 100% by weight of water, relative to the total weight of the composition.

30. The process according to claim 29, wherein the composition comprise from 0% to 80% by weight of water, relative to the total weight of the composition.

31. The process according to claim 30, wherein the composition comprises from 0% to 50% by weight of water, relative to the total weight of the composition.

32. The process according to claim 16, further comprising at least one adjuvant chosen from silicones in soluble, dispersed and microdispersed forms, nonionic, anionic, cationic and amphoteric surfactants, ceramides, glycoceramides and pseudoceramides, vitamins and provitamins, plant, animal, mineral and synthetic oils, waxes other than ceramides, glycoceramides and pseudoceramides, water-soluble and liposoluble, silicone-based and non-silicone-based sunscreens, nacreous agents and opacifiers, sequestering agents, plasticizers, solubilizers, acidifying agents, mineral and organic thickeners, antioxidants, hydroxy acids, penetrating agents, fragrances and preserving agents.

33. The process according to claim 16, wherein the composition is in a form of a cream, a liquid, and/or a gel comprising at least one thickener.
